(19) United States
(12) Patent Application Publication
(10) Pub. No.: US 2018/0348760 A1
(43) Pub. Date: Dec. 6, 2018

(54) Automatic Change Detection System

(71) Applicants: James Peverill, Milpitas, CA (US); Joel Michael Pedlikin, Brookline, MA (US); Justin McClellan, Boston, MA (US)

(72) Inventors: James Peverill, Milpitas, CA (US); Joel Michael Pedlikin, Brookline, MA (US); Justin McClellan, Boston, MA (US)

(21) Appl. No.: 15/609,058

(22) Filed: May 31, 2017

(51) Int. Cl. G05D 1/00; G01N 33/24; B64C 39/02; G01N 33/00; B63G 8/00; G01S 17/89; G01S 15/89; G01S 13/89; G01R 33/02

(52) U.S. Cl. CPC G05D 1/0088; G01N 33/246; B64C 39/024; G01N 33/0098; B63G 8/001; G01S 17/89; G05D 2201/0207; G01S 13/89; G01R 33/02; B63G 2008/004; B64C 2201/127; B64C 2201/141; G01S 15/89

(57) ABSTRACT

A system for tracking and reporting changes to a parcel of land comprising one or more stationary or mobile sensors mounted to fixed positions and/or unmanned vehicles, and sophisticated distributed network software for controlling the sensors and vehicles, analyzing the sensor data, and storing the data. The network software controls the sensor data gathering, the route any vehicles travel, and all the timing for data gathering. If unmanned vehicles are used, the routes and starting times can factor in weather, people and vehicle traffic, air traffic control information, and the previously data obtained sensor data from vehicle sensors on previous trips. Fixed sensors are environmentally protected and connected via wireless or wired signals. Vehicles can be stored in environmentally protective enclosures which can recharge or refuel the vehicles, and/or download any sensor data they obtained on previous surveys. Sensors carried can be imagers tuned to various parts of the electromagnetic spectrum, chemical detectors, radiation detectors, or magnetometers, and can be swapped out within the enclosure. The distributed network software processes and stores whatever data is obtained by the sensors and compares data to that obtained from previous sensor readings, identifying changes and reporting on those changes to users via a local display or any form of electronic communication relayed to another electronic device.

### CROSS-REFERENCE TO RELATED APPLICATIONS

This Application claims the benefit of priority under 35 U.S.C. § 119 to U.S. Provisional Application 62/323,572 filed on May 31, 2016, which is hereby incorporated by reference in its entirety.

### BACKGROUND OF THE DISCLOSED SUBJECT MATTER

### Field of the Disclosed Subject Matter

The disclosed subject matter relates to a system for tracking and reporting changes to or on a parcel of land. Particularly, the present disclosed subject matter relates to one or more sensors, either stationary or mounted on unmanned vehicles, and software which controls the sensors and vehicles as well as analyzes the data from these sensors.

### Description of Related Art

A variety of methods and systems are known for providing one-time images, sensor maps, and other data about land and its contents at narrow moments in time. Examples include photographs taken by spacecraft and manned aircraft, hand-held radiometer measurements, and data obtained by individual visits by unmanned vehicles.

Such conventional methods and systems generally have been considered satisfactory for their intended purpose. However, most land managers and ers are more interests d in the changes that occur to their land and its contents over time,rather than that land's specific state at one given time. For instance, farmers want to see the onset and growth of disease or pests, and detect increases and decreases in the soil moisture levels of their fields. Construction managers want to see the state of their site reparation and construction. Forest anagers want to see the arrival of disease, physical damage or fire. Ranchers want to monitor the number, location, and health of animals in their fields over time. Homeowners' associations want to identify new modifications to houses or yards. Military planners want to detect and track move ents of supplies, units and equipment in the local area and detect booby traps or improvised explosive devices.

As evident from the related art, conventional methods do not satisfy the above potential users, because they take ‘snapshots’ in time, rather than monitoring and identifying changes.

There thus remains a need for an efficient and economic method and system for effectively monitoring changes in land and its contents over time.

### SUMMARY OF THE DISCLOSED SUBJECT MATTER

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, the disclosed subject matter includes a method and system for monitoring land in order to track and report changes to that land. The invention uses some set of sensors attached to stationary mounts and/or unmanned vehicles to gather relevant information about the target land parcel. Network software gathers the data collected by these sensors and stores, processes, and analyzes it to identify and report on changes to the land parcel.

The disclosed subject matter also includes a system comprising multiple unmanned vehicles with sensors onboard, ltiple fixed sensors, and network based software, whereir the software is used to automatically control the vehicles, their onboard sensors, and the fixed sensors so as to obtain data on a parcel of land, and the network software stores and processes data from the sensors to detect, identify, and automatically report changes to that land.

The disclosed subject matter also includes a system where the above-mentioned sensors are mounted on poles, vehicles, or other stationary or mobile equipment.

The disclosed subject matter further includes a system where sensors are mounted on unmanned aerial vehicles (UAV), unmanned ground vehicles(UGV), or unmanned underwater vehicles(UUV), and where these unmanned vehicles can be permanently stored in one or more enclosures which may protect the vehicles from environmental damage.

The disclosed subject matter further includes a system where the sensors are visible, multi spectral, or infraredinfraredimaging cameras, chemical detectors, magnetometers, radiation detectors, or 3D scanning sensors such as Lidar, Radar, or acoustic sensors.

The disclosed subject matter further includes a system where the data from the sensors is transmitted to the network software using wireless or wired networks.

The disclosed subject matter further includes a system where the network software controls how frequently and/or where the sensors gather data, including a system where ground traffic, local sensor data, local weather conditions measured via sensors or communicated via a network, or Air Traffic Control information is used by the network software to automatically determine which vehicles should be deployed and/or at what times.

The disclosed subject matter further includes any system where the parcel of land being monitored is a golf course, farm, sports field, park or construction site.

The disclosed subject matter further includes a system where the sensors and vehicles are controlled so as to take data at the exact same times of day, each day.

The disclosed subject matter further includes a system where visible, infrared, or multispectral cameras are used to measure plant canopy temperature, detect the presence of disease or pests in plants, and/or measure soil moisture.

The disclosed subject matter further includes a system where the vehicle enclosure(s) automatically refuel or recharge the vehicle propulsion systems or download sensor data for processing after each vehicle has entered the enclosure.

The disclosed subject matter further includes a system where each vehicle's sensors can be automatically swapped while the vehicle is inside the enclosure.

The disclosed subject matter further includes a system where data is processed, analyzed and stored on distributed processing units in each enclosure, and a system where these processing units are connected via a network.

It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF THE DRAWINGS

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a schematic representation of the automatic change detection system. Unmanned vehicles (110) and (120) carry onboard sensors for tracking changes in land parcel (130). Fixed sensors (170) and (180) also track changes in land parcel (130). Sensors and vehicles are controlled via one or more network connected processing units (140), and upload whatever data they collect back to said processing unit. Unmanned vehicles may be stored in environmentally protective enclosures (150) and (160), which may also contain equipment to refuel or recharge the vehicles, upload sensor data they obtained, and/or process, analyze, or store that sensor data.

FIG. 2 is a schematic representation of a thermal imager and air temperature sensors on a UAV (210) being used to create estimated soil moisture maps, and a UGV (220) with a soil moisture probe (290) used to more accurately measure specific problem locations identified by those maps.

FIG. 3 is a schematic representation of a UAV (310) carrying visible and near-IR cameras commanded by control logic in central network software to survey a specific area every day in order to create normalized difference vegetation index (NDVI) maps that indicate plant health. Based on results of these maps the network software can then plan a follow-on survey flight(345) with the same UAV, where it flies lower and slower (315) over areas with significant decreases in plant health (335).

FIG. 4 is a schematic representation of fixed moisture sensors(470) providing Data to serve as ‘ground truth’ for estimated soil moisture maps created by a UAV (410) carrying a thermal camera.

### DETAILED DESCRIPTION OF AN EXEMPLARY EMBODIMENT

Reference will now be made in detail to exemplary embodiments of the disclosed subject matter, examples of which are illustrated in the accompanying drawings. The method and corresponding steps of the disclosed subject matter will be described in conjunction with the detailed description of the system.

The methods and systems presented herein may be used for automatic change detection. The disclosed subject matter is particularly suited for identifying changes in vegetation over time using a variety of sensors mounted on autonomous vehicles and/or fixed mounts.

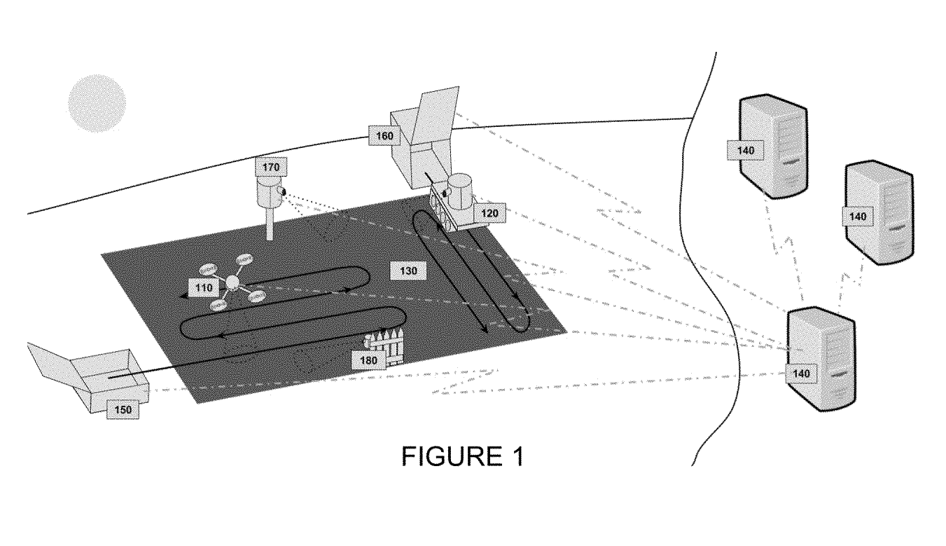

For purpose of explanation and illustration, and not limitation, an exemplary embodiment of the system in accordance with the disclosed subject matter is shown in FIG. 1 and is designated generally by reference character 100. Similar reference numerals (differentiated by the leading numeral) may be provided among the various views and Figures presented herein to denote functionally corresponding, but not necessarily identical structures.

As shown in FIG. 1, the system 100 generally operates over a generic parcel of land (130). Nothing is intended to be signified by the shape or color of the land parcel. It could be a private yard, park, golf course, farm, construction site, or any other piece of land. The system can include unmanned aerial vehicles (110), unmanned ground vehicles (120), network software (140), and environmentally protective enclosures for the vehicles (150, 160).

Sensors such as, but not limited to, visible imagers, infrared or multispectral imagers, chemical detectors, radiation detectors, and/or magnetometers are mounted to vehicles (110) and (120), and/or permanent fixtures (170) and (180), on the land parcel (130). Different or multiple sensors can be placed on different vehicles and fixtures. Sensors can operate during daylight hours, nighttime hours, or both.

Sensors are mounted to rigid fixtures (170) and (180) and can rotate and/or change elevation to monitor land close to them. Multiple sensors can be mounted to one fixture. A mounting fixture can be purpose-built for sensor mounting, or be an existing structure such as a house or a fence. Fixed sensors can transmit data to network software using satellite, cellular, or other wireless networks, or via a wired connection.

Sensors mounted to unmanned aerial vehicles (110) and/or unmanned ground vehicles (120) survey part or all of the parcel of land (130) as the vehicle they are mounted to steers a path over the parcel.

Unmanned vehicles (110) and (120) can be stored inside environmentally protective enclosures (150) and (160), depart the enclosures in order to steer the desired survey path, then travel back inside the enclosures to be stored until it is time to perform another survey. One enclosure can store multiple unmanned vehicles. Enclosures can function as mounting fixtures for sensors in addition to their vehicle storage function.

Unmanned Vehicles (110) and (120) can directly transmit data back to the network software using satellite, cellular, or other wireless networks. Alternately, once they are back inside enclosures (150) and (160), the vehicles can transfer the data obtained to the enclosures, which can be capable of transmitting the data back to the network software either wirelessly or via a wired connection.

Enclosures (150) and (160) can include the capability to recharge or refuel unmanned vehicles (110) and (120) while they are stored. Enclosures can include the capability to swap out sensors on the vehicles.

Enclosures (150) and (160) can include the capability to store data obtained by sensors on unmanned vehicles (110) and (120), as well as the capability to process and analyze that data. Enclosures can be networked together to jointly store, process, and analyze sensor data.

Network software (140) can use data previously taken from fixed and mobile sensors, as well as outside data such as weather services, ground traffic reports or air traffic control, to plan fixed and mobile sensor data gathering.

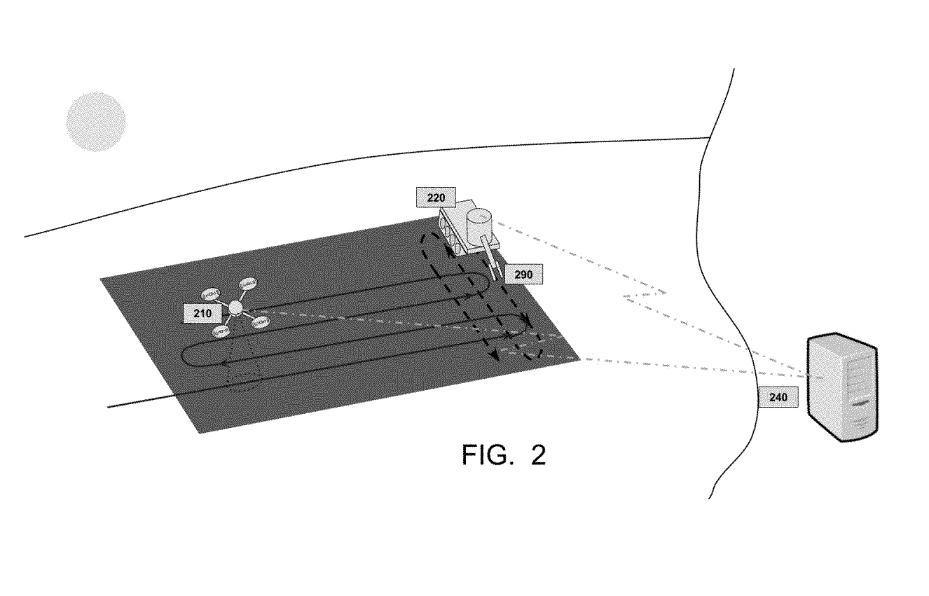

In accordance with another embodiment of the disclosed subject matter, shown in FIG. 2, a highly accurate, calibrated thermal imager and air temperature sensors on a UAV (210) can provide data which can be combined to create estimated soil moisture maps. Network software (240) can then compare multiple soil moisture maps which were generated over time to identify where moisture levels are declining Network software(240) can analyze these maps to command a UGV(220) with a soil moisture probe(290) to travel to critical locations and measure soil moisture levels directly in situ. This data can then be sent back to the network software (240) and superimposed on the soil moisture map or used to recalibrate the soil moisture map, i.e. used to adjust all the previously estimated values on that map to be more accurate based on these in situ values.

In accordance with another embodiment of the disclosed subject matter, data from fixed sensors can be used by network software to plan when mobile vehicles begin surveys of land, what sensors they should carry to survey that land, and where they should travel.

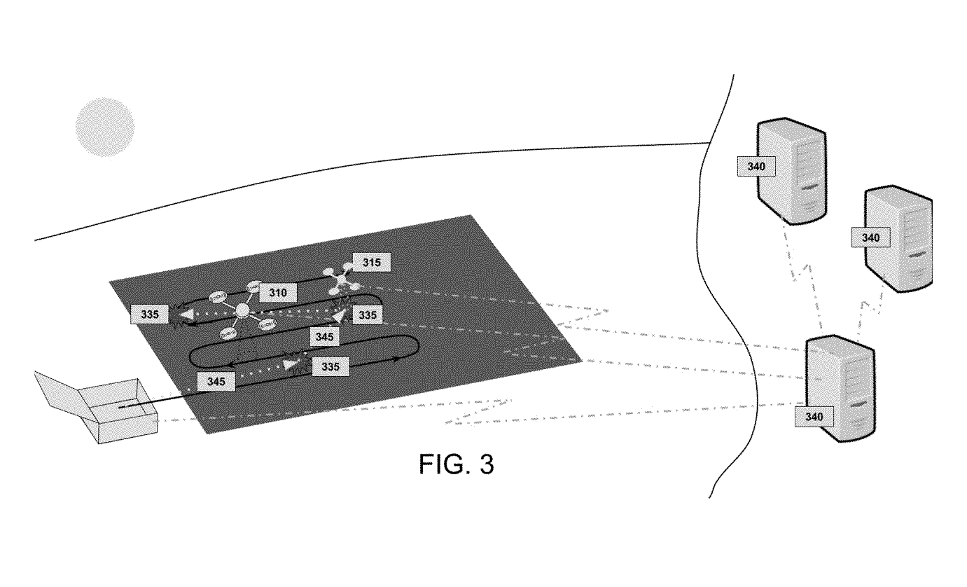

In a specific example of this embodiment shown in FIG. 3, UAV (310) is commanded by control logic in central network software(340) to survey a specific area every day (for instance a golf course or farm), using a near-IR and visible camera to create normalized difference vegetation index (NDVI) maps that indicate plant health. If and when an algorithm within the network software identifies significant negative changes in plant health(335) on these maps, the network software alerts the landowner as to the magnitude of the change and the location of the plants whose health is declining The network software can then plan a follow-on survey flight(345) with the same UAV, where it flies lower and slower (315) over areas with significant decreases in plant health (335) to get higher resolution imagery. Part of planning this follow-on survey flight might be swapping out the visible or near-IR cameras on the UAV for multispectral cameras ‘tuned’ to specific wavelengths that are useful for identifying specific diseases or pests in the vegetation being imaged.

In another specific example of this embodiment, a rain gauge could be used by network software to determine when it is safe for a UAV to fly, i.e. wait until rain ends before commanding a UAV to take off.

In another specific example of this embodiment, a wind sensor (anemometer) could be used by network software to determine if winds are too high for a UAV to fly, and command an autonomous ground vehicle to immediately perform a less extensive survey instead.

In another specific example of this embodiment, light sensors could be used by network software to determine if the light levels are sufficiently high for UAV-based cameras to be able to take high-quality images, and command that UAV when to take off.

In another specific example of this embodiment, simple motion sensors could be used by network software to make sure that no people are on a plot of land so that it is safe for an autonomous vehicle to survey that plot of land on the ground or from the air.

In another specific example of this embodiment, fixed radiation or chemical sensors could be used by network software to create a flight path for a UAV or UGV that investigates the presence of radiation or harmful chemicals more thoroughly than fixed sensors can.

In accordance with another embodiment of the disclosed subject matter, data from fixed sensors and/or sensors on mobile vehicles can be combined by network software to provide immediate and more useful information to a land owner.

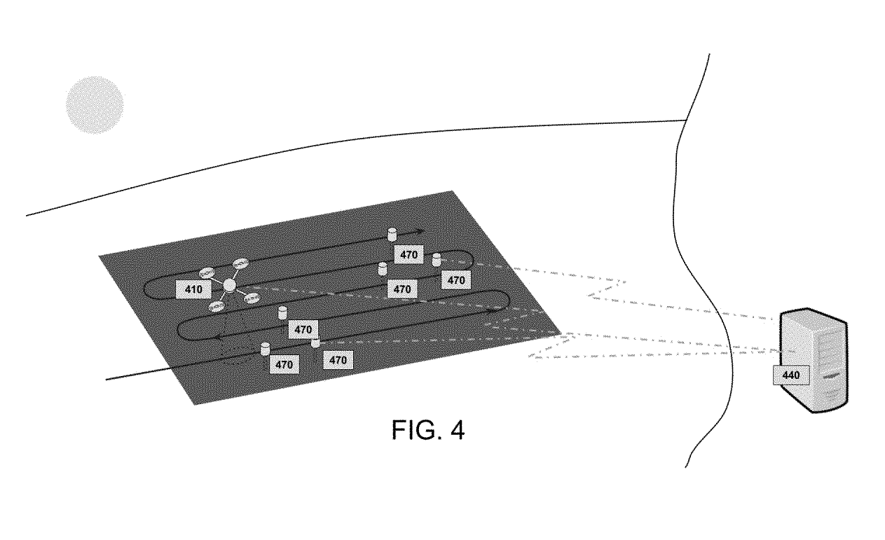

In a specific example of this embodiment, shown in FIG. 4, data from fixed, buried soil moisture sensors (470) can serve as ‘ground truth’ for estimated soil moisture maps created by a UAV (410) surveying a golf course or farm with a thermal imager. Anywhere from one to hundreds of soil moisture sensors can be placed at the most sensitive locations on the golf course or farm, for instance between one and three such sensors could be placed on each of the 18 greens on a golf course. These sensors directly measure the moisture level in the soil as a percentage, but only provide accurate information within a few feet of their location. Both the moisture measurements and the precise locations are provided to the network software (440). The UAV (410), on the other hand, uses a thermal camera to create a map that estimates the soil moisture levels across the entire golf course or farm. The network software can then adjust/calibrate each estimated soil moisture map based on the directly measured soil moisture levels from the buried moisture sensors. This process can be repeated multiple times a day when soil moisture levels are critical (such as during a drought), or much less frequently when soil moisture levels are less important.

In another specific example of this embodiment, image data from UGV's driving underneath canopies and UAV's flying above canopies can be combined to estimate yield from fruit or nut trees, grape vines, or other crops. Centralized network software controls the movements and routes of the UGV and UAV, optimizing the times that they take their measurements based on weather, light levels, and shadowing. That software also commands more frequent imaging as harvesting time approaches.

In accordance with another embodiment of the disclosed subject matter, A UAV carrying both visible and near-IR imaging cameras could use images from those cameras to create NDVI maps to monitor the health of crops on a farm, and network software could then roughly estimate potential plant yields using an algorithm combining that health data and plant shadow lengths (to approximate plant height). Once the yield estimate reached a certain point, the network software could command that the UAV's cameras be swapped out for a LIDAR, which the UAV would fly with and use to survey plant heights more precisely.

While the disclosed subject matter is described herein in terms of certain preferred embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. A system comprising: multiple unmanned vehicles with sensors onboard, multiple fixed sensors, and network based software, wherein the software is used to automatically control the vehicles, their onboard sensors, and the fixed sensors so as to obtain data on a parcel of land, and the network software stores and processes data from the sensors to detect, identify, and automatically report changes to that land.

2. The system of claim 1 wherein the sensors are mounted on poles, vehicles, or other stationary or mobile equipment

3. The system of claim 1 wherein the sensors are mounted on unmanned aerial vehicles, unmanned ground vehicles or unmanned underwater vehicles

4. The system of claim 1 wherein the sensors are visible, multi spectral, or infrared imaging cameras

5. The system of claim 1 wherein the sensors include chemical detectors

6. The system of claim 1 wherein the sensors include magnetometers

7. The system of claim 1 wherein the sensors include radiation detectors

8. The system of claim 1 wherein the sensors include 3D scanning sensors such as Lidar, Radar, or acoustic sensors.

9. The system of claim 1 wherein the data from the sensors is transmitted to the network software using wireless or wired networks.

10. The system of claim 3 wherein one or more of the unmanned vehicles are permanently stored in one or more enclosures which may protect the vehicle from environmental damage

11. The system of claim 1 wherein the network software controls how frequently and/or where the sensors gather data

12. The system of claim 1 wherein ground traffic, local sensor data, local weather conditions measured via sensors or communicated. via a network, or Air Traffic Controlinformation is used by the network software to automatically determine which vehicles should be deployed and/or at what times

13. The system of claim 1 wherein the parcel of land is a golf course, farm, sports field, park, or construction site

14. The system of claim 1 wherein the sensors and vehicles are controlled so as to take data at the exact same times of day, each day.

15. The system of claim 4 wherein the cameras are used to measure plant canopy temperature

16. The system of claim 4 wherein the cameras are used to detect the presence of disease or pests in plants on the land parcel

17. The system of claim 4 wherein the sensors measure soil moisture

18. The system of claim 10 wherein the enclosure(s) automatically refuel or recharge the vehicle propulsion systems

19. The system of claim 10 wherein the enclosure(s) download sensor data for processing after each vehicle has entered the enclosure

20. The system of claim 10 wherein each vehicle's sensors can be automatically swapped while the vehicle is inside the enclosure

21. The system of claim 10 where data is processed, analyzed and stored on distributed processing units in each enclosure

22. The system of claim 21 where the processing units are connected via a network
